(19) United States
(12) Patent Application Publication
(10) Pub. No.: US 2015/0265202 A1
(43) Pub. Date: Sep. 24, 2015

(54) METHOD FOR COLLECTING DUODENAL JUICE

(71) Applicant: OLYMPUS MEDICAL SYSTEMS CORP., Tokyo (JP)

(72) Inventor: Mari NAKATA, Tokyo (JP)

(73) Assignee: OLYMPUS MEDICAL SYSTEMS CORP., Tokyo (JP)

(21) Appl. No.: 14/220,250

(22) Filed: Mar. 20, 2014

(51) Int. Cl. A61B 5/00; A61B 1/273; A61B 1/015; A61B 10/00

(52) U.S. Cl. CPC A61B 5/4283; A61B 5/4216; A61B 10/0045; A61B 1/2736; A61B 1/015; A61B 2010/0061

(57) ABSTRACT

A method for collecting duodenal juice containing bile under an endoscope is provided. In the method, an endoscope is inserted into a duodenum of a subject from which collection of duodenal juice is performed. In a state in which an illuminating section of the endoscope is stopped at the duodenum, an illumination light is generated. The illumination light has a specific wavelength band capable of displaying a component including bile in an enhanced manner, within a visible wavelength region. By an imaging section of the endoscope, a subject image is captured. The subject image is illuminated by the illumination light of the specific wavelength band. Based on an output signal from the imaging section, a subject information image expressing subject information is displayed in a display section. In the displayed subject information image, duodenal juice containing bile is displayed in an enhanced manner. Then, the duodenal juice is collected.

### BACKGROUND

1. Technical Field

The present invention relates to a method for collecting duodenal juice. In particular, the present invention relates to a method for collecting duodenal juice containing bile under an endoscope.

2. Related Art

Duodenal juice is a mixed body fluid containing pancreatic juice discharged from the pancreas, bile discharged from the bile duct, and mucus secreted in the duodenal juice. Pancreaticobiliary diseases can be detected by the duodenal juice being collected and biomolecules in the duodenal juice being analyzed. For example, the specification of US Patent Application Publication No. 2012/0258478 describes a method for collecting duodenal juice to detect pancreaticobiliary diseases. In the method, a stimulant of pancreatic juice secretion is administered to a subject from which the duodenal juice is discharged. Sampling equipment is disposed in the duodenum of the subject. Collection of the duodenal juice using the sampling equipment is started within five minutes of the administration of the stimulant of pancreatic juice secretion. Collection of the duodenal juice is completed when the collection period of the duodenal juice reaches five minutes since the start of collection. In this method, duodenal juice enabling testing for pancreatic diseases can be collected with certainty in a short amount of time.

Bile contains bilirubin. Therefore, duodenal juice containing bile may exhibit a light yellow color (including yellowish colors such as yellow to brownish-yellow) as a whole. If the light yellow fluid can be identified under an endoscope, the duodenal juice containing bile can be collected. However, the light yellow fluid is ordinarily difficult to identify under an endoscope. Targeting and collecting the duodenal juice containing bile that exhibits the yellow color is difficult.

In relation to the foregoing, US Patent Application Publication No. 2011/0230712 specification has a description related to observation mode control in instances in which the presence of cancer is examined or cancer is treated under an endoscope. In addition to a normal light observation mode, a plurality of narrow-band light observation modes are used as the observation modes in these instances.

In the normal light observation mode, white light is illuminated onto a target site (subject) inside the body of the subject. As a result, a subject image is obtained that is substantially similar to macroscopic observation. On the other hand, in the narrow-band light observation mode, light having a narrower band (narrow-band light) than the illumination light used in the normal light observation mode is illuminated onto the subject. As a result, a subject image is obtained in which blood vessels in the superficial portion of the mucous membrane in the body and the like are enhanced. The narrow-band light observation modes include a first narrow-band light observation mode and a second narrow-band light observation mode. The first narrow-band observation mode prioritizes cancer identification. The second narrow-band observation mode enables cancer identification while preventing bile from being mistakenly recognized as a hemorrhage.

### SUMMARY

It is thus desired to provide a method for collecting duodenal juice that can enable duodenal juice containing bile to be easily identified under an endoscope and efficiently collected.

According to an exemplary embodiment of the present disclosure, a method for collecting duodenal juice is provided. The method for collecting duodenal juice includes: a first step of inserting an endoscope into a duodenum of a subject from which collection of duodenal juice is performed; a second step of generating an illumination light of a specific wavelength band, within a visible wavelength region, that is capable of displaying a component including bile in an enhanced manner, the illumination light of the specific wavelength band being generated in a state in which an illuminating section of the endoscope is stopped at the duodenum; a third step of capturing a subject image that is illuminated by the illumination light of a specific wavelength band, the subject image being captured by an imaging section of the endoscope; a fourth step of displaying a subject information image in a display section based on an output signal from the imaging section, the subject information image expressing subject information; and a fifth step of collecting duodenal juice containing the bile that is displayed in an enhanced manner in the subject information image displayed in the display section.

### BRIEF DESCRIPTION OF THE DRAWINGS

In the accompanying drawings:

FIG. 1 is a diagram of an overall configuration of an endoscopy system used to perform a method for collecting duodenal juice according to an embodiment;

FIG. 2 is a block diagram of an internal configuration of the endoscopy system shown in FIG. 1;

FIG. 3 is a diagram of a configuration of a rotary filter shown in FIG. 2;

FIG. 4 is a graph showing spectral characteristics of a first filter set of the rotary filter shown in FIG. 3;

FIG. 5 is a graph showing spectral characteristics of a second filter set of the rotary filter shown in FIG. 3; FIG. 6 is a block diagram of a configuration of sampling equipment shown in FIG. 1;

FIG. 7 is a flowchart of procedures in the method for collecting duodenal juice using the endoscopy system shown in FIG. 1;

FIG. 8 is a diagram for describing a collection position for duodenal juice containing bile; and

FIG. 9A is a diagram of an endoscopic image of the duodenal juice containing bile in a normal light observation mode, and FIG. 9B is a diagram of an endoscopic image of the duodenal juice containing bile in a narrow-band light observation mode.

### DESCRIPTION OF THE EMBODIMENTS

An embodiment of a method for collecting duodenal juice of the present invention will be described with reference to the drawings.

### (Endoscopy System)

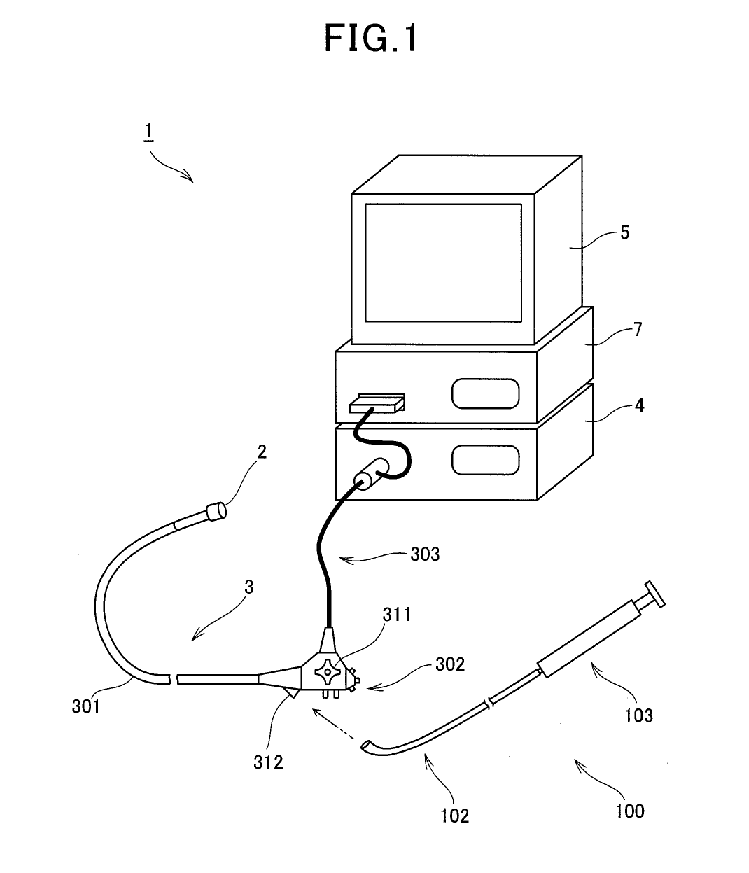

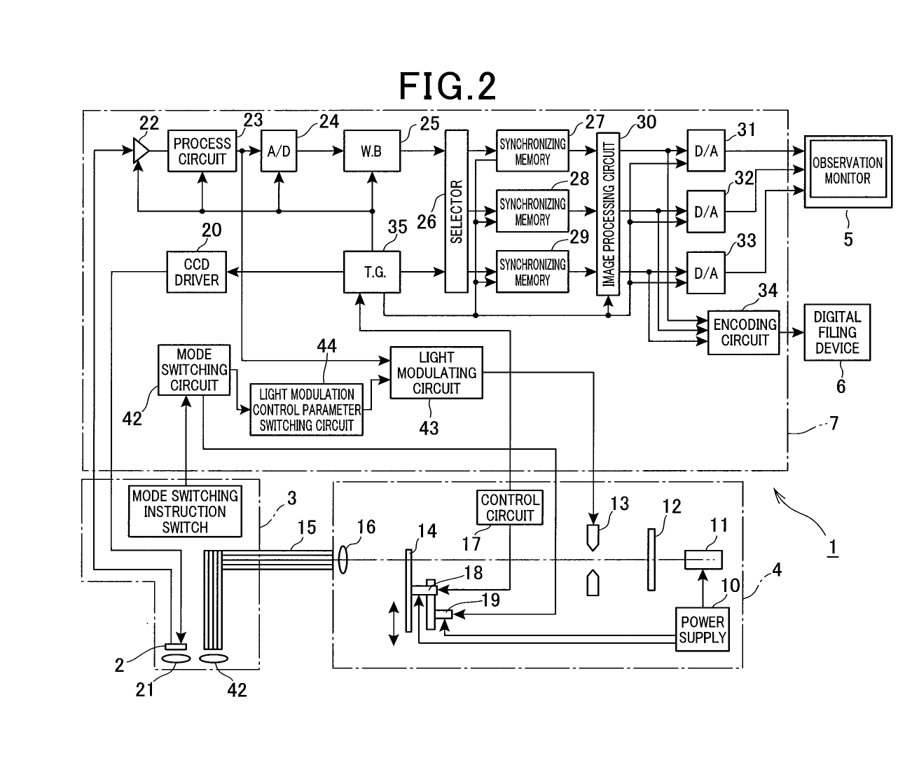

FIG. 1 shows an overall configuration of an endoscopy system 1 used to perform the method for collecting duodenal juice according to the present embodiment. FIG. 2 shows an internal configuration of the endoscopy system 1.

An endoscope device (for example, refer to JP-B2-4384626) having a plurality of observation modes is applied to the endoscopy system 1 used according to the present embodiment. The plurality of observation modes include: 1) a mode in which a target site (subject) inside the body of a subject is illuminated with white light and observed (hereinafter referred to as “normal light observation mode”); and 2) a mode in which the target site (subject) is illuminated with light (hereinafter referred to as “narrow-band light”) having two wavelengths (blue light: 390 nm to 445 nm [corresponding to the first wavelength band]; and green light: 530 nm to 550 nm [corresponding to the second wavelength band]) that are set to narrow bands easily absorbed by the hemoglobin within the blood (hereinafter referred to as “narrow-band light observation mode”).

As shown in FIG. 1, the endoscopy system 1 includes an electronic endoscope 3, a light-source device 4, an observation monitor (display section) 5, and a video processor 7. The electronic endoscope 3 has a charge-coupled device (CCD) (imaging section) 2 that is inserted into the body of the subject. The CCD 2 then captures an image of the target site (subject). The light-source device 4 supplies the electronic endoscope 3 with light for illumination. In addition, peripheral devices, such as a digital filing device 6 shown in FIG. 2, are mounted in the endoscopy system 1. Furthermore, according to the present embodiment, sampling equipment 100 is also used in the endoscopy system 1. The sampling equipment 100 is used to collect the duodenal juice.

### (Electronic Endoscope)

A commonly used endoscope can be applied as the electronic endoscope 3 shown in FIG. 1. For example, a side-view endoscope often used in the biliopancreatic region or an upper gastrointestinal endoscope used to examine the stomach and the duodenum can be used. The insertion path of the endoscope may be oral or nasal.

The electronic endoscope 3 has an inserting section 301, a manipulating section 302, and a universal cable 303. The inserting section 301 is formed into a thin, elongated tube shape and can be inserted into the body of the subject. The manipulating section 302 is provided on a rear end side of the inserting section 301. The universal cable 303 configures a connecting section that extends from the manipulating section 302. The electronic endoscope 3 is detachably connected to the light-source device 4 and the video processor 7 by the universal cable 303.

The inserting section 301 has a tip portion, curved portion, and a flexible portion (soft portion). The curved portion is provided at the rear end of the tip portion and can be bent freely. The flexible portion extends from the rear end side of the curved portion to the front end side of the manipulating section 302. In addition to the CCD 2, an illumination lens (illuminating section) 42, an object lens (configuring the imaging section together with the CCD 2) 21, and the like are disposed at the tip portion. The illumination lens 42 irradiates light. Reflected light of the irradiated light is incident on the object lens 21. The object lens 21 then forms an image on the CCD 2. The curved portion can be curved upward, downward, and to the left and right by an angle knob 311 being manipulated. The angle knob 311 is provided in the manipulating section 302.

A light guide 15 (see FIG. 2) is inserted into the inserting section 301. The light guide 15 transmits light. An incidence side of the light guide 15 is connected to the light-source device 4 by the universal cable 303. The light from the light-source device 4 is transmitted through the light guide 15 and emitted forward from the tip portion. The light is then illuminated onto the target site inside the body of the subject. Returned light from the target site that is illuminated by the illumination light is formed into an image on the CCD 2 by the object lens 21 at the tip portion. The returned light is photoelectrically converted by the CCD 2. The CCD 2 then outputs the returned light as a signal to the video processor 7. The signal corresponds with the amount of electric charge collected based on the intensity (light quantity) of the incident light. The video processor 7 performs signal processing on the outputted signal. The video processor 7 then supplies the processed signal to the observation monitor 5 as a standard video signal (image signal).

A treatment tool insertion opening 312 is provided on a front end side of the manipulating section 302. The treatment tool insertion opening 312 communicates with a channel within the inserting section 301. During a biopsy procedure and the like, a treatment tool such as biopsy forceps is inserted into the treatment tool insertion opening 312. The treatment tool then passes through the channel within the inserting section 301 and is projected forward from an opening (forceps opening) provided in the tip portion of the inserting section 301.

According to the present embodiment, during collection of the duodenal juice, the sampling equipment 100 is inserted into the treatment tool insertion opening 312. The sampling equipment 100 then passes through the channel within the inserting section 301 and is projected forward from the opening at the tip portion of the inserting section 301.

An observation mode switching instruction switch 41 is provided in the manipulating section 302. The observation mode switching instruction switch 41 is used to give an instruction to switch the observation mode between a plurality of observation modes including the normal light observation mode and the narrow-band light observation mode set in the endoscopy system 1 in advance. The observation mode switching instruction switch 41 is connected to the video processor 7 by the universal cable 303.

### (Light-Source Device)

The light-source device 4 includes a power supply section 10, a xenon lamp 11, a heat ray cut-off filter 12, a beam limiting device 13, a rotary filter 14, a condenser lens 16, a control circuit 17, a rotary filter motor 18, and a mode switching motor 19. The power supply section 10 supplies power to the xenon lamp 11, the beam limiting device 13, a rotary filter motor 18, and the mode switching motor 19.

Heat rays of the white light emitted from the xenon lamp 11 are blocked by the heat ray cut-off filter 12. The light quantity of the white light is controlled by the beam limiting device 13. The white light is turned into frame sequential light by the rotary filter 14, under rotation control by the control circuit 17. The frame sequential light is concentrated onto an incidence surface of the light guide 15, via the condenser lens 16.

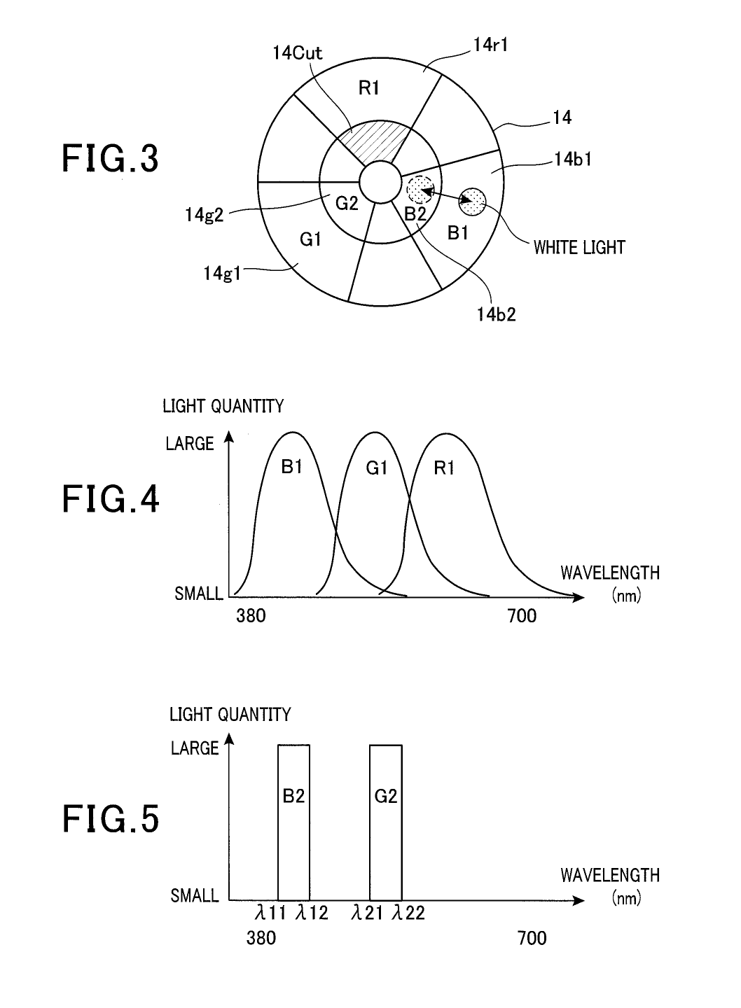

As shown in FIG. 3, the rotary filter 14 is formed into a circular disk shape. The rotary filter 14 rotates with its center as the rotation axis. The rotary filter 14 has a dual structure in which the filter configuration differs between an outer portion and an inner portion in a radial direction.

In the dual structure, a first filter set is disposed on the outer portion in the radial direction. The first filter set is for the normal light observation mode. As shown in FIG. 4, the first filter set is configured such that an R1 filter portion 14r1, a G1 filter portion 14g1, and a B1 filter portion 14b1 are disposed in the circumferential direction of the rotary filter 14. The R1 filter portion 14r1 transmits the red wavelength region. The G1 filter portion 14g1 transmits the green wavelength region (G1). The B1 filter portion 14b1 transmits the blue wavelength region (B1).

As a result, in the normal light observation mode, the white light incident onto the rotary filter 14 from the beam limiting device 13 passes through the filter portions 14r1, 14g1, and 14b1 of the first filter set. The white light is emitted from the rotary filter 14 as frame sequential light having spectral characteristics suitable for color reproduction and in which red (R1), green (G1), and blue (B1) overlap, as shown in FIG. 4.

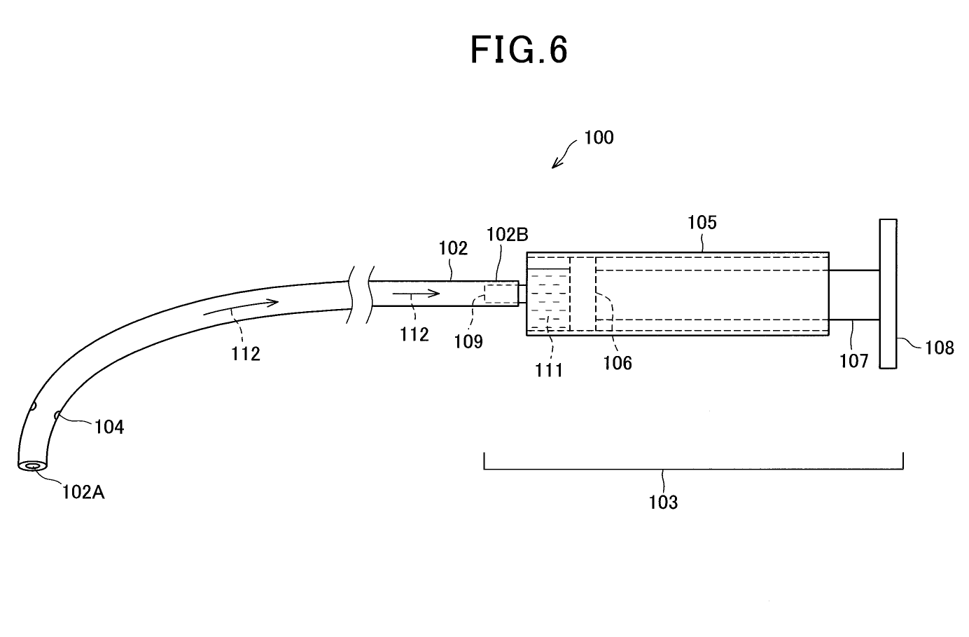

On the other hand, in the dual structure, a second filter set is disposed in the inner portion in the radial direction. The second filter set is for the narrow-band light observation mode. As shown in FIG. 5, the second filter set is configured such that a G2 filter portion 14g2, a B2 filter portion 14b2, and a light-blocking filter portion 14Cut are disposed in the circumferential direction of the rotary filter 14. The G2 filter portion 14g2 transmits light in the green (G2) wavelength region λ11 to λ12. The B2 filter portion 14b2 transmits light in the blue (B2) wavelength region λ21 to λ22. The light-blocking filter portion 14Cut blocks light.

According to the present embodiment, the wavelength region κ21 to λ22 of the B2 filter portion 14b2 is 395 nm to 445 nm. The wavelength region λ11 to λ12 of the G2 filter portion 14g2 is 530 nm to 550 nm. The light of these two wavelength regions is easily absorbed by the hemoglobin in the blood.

As a result, in the narrow-band light observation mode, the white light incident onto the rotary filter 14 from the beam limiting device 13 passes through the filter portions 14g2, 14b2, and 14Cut of the second filter set. The white light is emitted from the rotary filter 14 as frame sequential light of two narrow bands having discrete spectral characteristics of green (G2) and blue (B2) as shown in FIG. 5.

The rotary filter motor 18 rotates the rotary filter 14 under drive-control by the control circuit 17. The mode switching motor 19 moves the rotary filter 14 in the radial direction (direction perpendicular to the light path of the rotary filter 14) based on a control signal from a mode switching circuit 42 within the video processor 7. As a result, the mode switching motor 19 selectively moves the first filter set or the second filter set onto the light path.

The light-source device 4 may use a semiconductor light-emitting device (such as a light-emitting diode or a semiconductor laser diode) as the light source, instead of the xenon lamp 11. Alternatively, light having the wavelength used in the narrow-band light observation mode may be obtained by the semiconductor light-emitting device or a combination of the semiconductor light-emitting device and a fluorescent body.

### (Video Processor)

The video processor 7 performs signal processing on an imaging signal from the CCD 2 of the electronic endoscope 3 and displays an endoscopic image on the observation monitor 5. In addition, the video processor 7 encodes the endoscopic image and outputs the encoded endoscopic image to the image filing device 6 as a compressed image.

Specifically, as shown in FIG. 2, the video processor 7 includes a CCD driver circuit 20 that drive-controls the CCD 2. In addition, the video processor 7 includes an amplifier 22, a process circuit 23, an analog/digital (AD) converter 24, a white-balance circuit 25, a selector 26, synchronizing memories 27, 28, and 29, an image processing circuit 30, digital/analog (D/A) converters 31, 32, 33, an encoding circuit 34, a timing generator 35, the mode switching circuit 42, a light modulating circuit 43, and a light modulation control parameter switching circuit 44.

The amplifier 22 amplifies the image signal that has been photoelectrically converted by the CCD 2. The amplifier 22 then outputs the amplified image signal to the process circuit 23.

The process circuit 23 performs processing operations such as correlative double sampling and noise removal on the amplified image signal. The process circuit 23 then outputs the processed image signal to the A/D converter 24.

The A/D converter 24 converts the image signal from the process circuit 23 from an analog signal to a digital signal. The A/D converter 24 then outputs the digital signal to the white-balance circuit 25 as image data.

The white-balance circuit 25 performs a white-balance processing operation on the image data from the A/D converter 24. The white-balance circuit 25 then outputs the processed image data to the selector 26.

The selector 26 selects the image data from the white-balance circuit 25 and outputs the image data to the synchronizing memories 27, 28, and 29 corresponding with the frame sequential light for each filter portion of the rotary filter 14 (the filter portions 14r1, 14g1, and 14b1 for the first filter set, and the filter portions 14g2, 14b2, and 14Cut for the second filter set).

The synchronizing memories 27, 28, and 29 temporarily store the image data from the selector 26. The synchronizing memories 27, 28, and 29 then synchronously read out the image data and output the image data to the image processing circuit 30.

The image processing circuit 30 performs image processing operations, such as gamma correction processing, edge enhancement processing, and color processing on each piece of image data of the frame sequential light that has been synchronously read out. The image processing circuit 30 then outputs color image data for R, G and B to the respective D/A converters 31, 32, and 33, and the encoding circuit 34.

The D/A converters 31, 32, and 33 convert the color image data for R, G, and B from the image processing circuit 30 to analog signals. The D/A converters 31, 32, and 33 then output the analog signals to the observation monitor 5 as video signals.

The encoding circuit 34 encodes the color image data for R, G, and B from the image processing circuit 30. The encoding circuit 34 then outputs the encoded color image data for R, G, and B to the digital filing device 6.

The timing generator 35 receives input of a synchronizing signal from the control circuit 17 of the light-source device 4. The synchronizing signal is synchronized with the rotation of the rotary filter 14. The timing generator 35 then outputs various timing signals based on the synchronizing signal to the above-described circuits.

The mode switching circuit 42 receives a mode switching instruction signal from the mode switching instruction switch 41. When the mode switching instruction signal is received, the mode switching circuit 24 outputs control signals based on the mode switching instruction signal to the light modulating circuit 43, the light modulation control parameter switching circuit 44, and the mode switching motor 19 of the light-source device 4.

The light modulation control parameter switching circuit 44 outputs light modulation control parameters for the first filter set or the second filter set of the rotary filter 14 to the light modulating circuit 43, based on the control signal from the mode switch circuit 42.

The light modulating circuit 43 controls light modulation of the beam limiting device 13 to achieve appropriate brightness, based on the control signal from the mode switching circuit 42 and the light modulation control parameters from the light modulation control parameter switching circuit 44.

### (Operation in Normal Light Observation Mode)

In the normal light observation mode, the mode switching motor 19 is controlled by the control signal from the mode switching circuit 42 such that the first filter set of the rotary filter 14 is positioned on the light path of the light-source device 4. Then, the video processor 7 performs synchronization and signal processing of the image signal picked up by the CCD 2. The video processor 7 generates color image data (R, G, and B) of the three components, red (R), green (G), and blue (B). The video processor 7 displays a subject information image expressing subject information based on the color image data on the observation monitor 5.

Here, as shown in FIG. 4, the wavelength regions of the R1 filter portion 14r1, the G1 filter portion 14g1, and the B1 filter portion 14b1 configuring the first filter set overlap.

Therefore, an endoscopic image expressing the subject information in natural color reproduction can be obtained from the image data (R), the image data (G), and the image data (B). The image data (R) is obtained by the CCD 2 capturing returned light during irradiation of red light that passes through the R1 filter portion 14r1. The image data (G) is obtained by the CCD 2 capturing returned light during irradiation of green light that passes through the G1 filter portion 14g1. The image data (B) is obtained by the CCD 2 capturing returned light during irradiation of blue light that passes through the B1 filter portion 14b1.

### (Operation in Narrow-Band Light Observation Mode)

In the narrow-band light observation mode, the mode switching instruction switch 41 is manipulated. The signal from the mode switching instruction switch 41 is inputted into the mode switching circuit 42 of the video processor 7. The mode switching circuit 42 outputs a control signal to the mode switching motor 19. As a result, the mode switching motor 19 drives the rotary filter 14 in relation to the light path such as to move the first filter set of the rotary filter 14 that is on the light path in the normal light observation mode. The mode switching motor 19 then disposes the second filter set (G2 filter portion 14g2, B2 filter portion 14b2, and light-blocking filter portion 14Cut) on the light path.

At this time, the transmitted light quantity of the second filter set becomes less than the transmitted light quantity of the first filter set, because the second filter set passes a narrower band of light. Therefore, the light modulation control parameter switching circuit 44 outputs light modulation control parameters adhering to the first filter set or the second filter set of the rotary filter 14 to the light modulating circuit 43. As a result, the light modulating circuit 43 controls the beam limiting device 13. Therefore, image data that has sufficient brightness can be obtained even in the narrow-band light observation mode.

The image processing circuit 30 generates color image data (R₁, G₁, and B₁) of the three components R, G, and B from the image data (G) and the image data (B). The image data (G) is obtained by the CCD 2 capturing returned light during irradiation of green narrow-band light that passes through the G2 filter portion 14g2. The image data (B) is obtained by the CCD 2 capturing returned light during irradiation of blue narrow-band light that passes through the B2 filter portion 14b2.

Specifically, the color image data (R₁) corresponding to red is generated from the image data (G) obtained during irradiation of the green narrow-band light. The color image data (G₁) corresponding to green and the color image data (B₁) corresponding to blue are generated from the image data (B) obtained during irradiation of the blue narrow-band light.

In other words, the image processing circuit 30 generates the color image data (R₁, G₁, and B₁) of the three components R, G, and B from the image data (G) obtained during irradiation of the green narrow-band light and the image data (B) obtained during irradiation of the blue narrow-band light, by the following expression (1).

$$( \begin{matrix}{R}_{1} \\ {G}_{1} \\ {B}_{1}\end{matrix} ) = ( \begin{matrix}h 11 & h 12 \\ h 21 & h 22 \\ h 31 & h 32\end{matrix} ) ( \begin{matrix}G \\ B\end{matrix} ) = ( \begin{matrix}h 11 \cdot G + h 12 \cdot B \\ h 21 \cdot G + h 22 \cdot B \\ h 31 \cdot G + h 32 \cdot B\end{matrix} ) \qquad K ( 1 )$$

Here, h11 and h12 are coefficients for the color image data (R₁) corresponding with red. h21 and h22 are coefficients for the color image data (G₁) corresponding with green. h31 and h32 are coefficients for the color image data (B₁) corresponding with blue.

According to the present embodiment, for example, h11=1, h12=0, h21=0, h22=1.2, h31=0, and h32=0.8 can be used as the values of the coefficients. In this instance, when the coefficient values are substituted in the above-described expression (1), the following expression (2) can be obtained.

$$( \begin{matrix}{R}_{1} \\ {G}_{1} \\ {B}_{1}\end{matrix} ) = ( \begin{matrix}1 & 0 \\ 0 & 1.2 \\ 0 & 0.8\end{matrix} ) ( \begin{matrix}G \\ B\end{matrix} ) = ( \begin{matrix}1 \cdot G + 0 \cdot B \\ 0 \cdot G + 1.2 \cdot B \\ 0 \cdot G + 0.8 \cdot B\end{matrix} ) = ( \begin{matrix}1 \cdot G \\ 1.2 \cdot B \\ 0.8 \cdot B\end{matrix} ) \qquad K ( 2 )$$

In other words, in the color image data (R₁, G₁, and B₁) of the three components R, G, and B, the color image data (R₁) corresponding to red is calculated by the product of the image data (G) obtained during irradiation of the green narrow-band light and the coefficient h11(=1). In addition, the color image data (G₁) corresponding to green is calculated by the product of the image data (B) obtained during irradiation of the blue narrow-band light and the coefficient h22(=1.2). Furthermore, the color image data (B₁) corresponding to blue is calculated by the product of the image data (B) obtained during irradiation of the blue narrow-band light and the coefficient h32(=0.8).

As a result, the color image data (R₁, G₁, and B₁) of the three components R, G, and B is generated. The subject information image expressing the subject information based on the color image data is displayed on the observation monitor 5. In the subject information image, capillaries and deep blood vessels in the superficial portion of the mucous membrane and the like are displayed in an enhanced state. The reason for the enhanced display is that, within the visible wavelength region, the blue narrow-band light has a short wavelength. The blue narrow-band light is strongly absorbed by the capillaries in the shallow portion of the mucous membrane. The blue narrow-band light is strongly reflected and dispersed by portions other than the capillaries. On the other hand, the green narrow-band light has a longer wavelength than the blue narrow-band light. The green narrow-band light reaches deeper into the mucous membrane than the blue narrow-band light and is strongly absorbed by the blood vessels in this portion. The green narrow-band light is strongly reflected and dispersed by portions other than the blood vessels.

### (Sampling Equipment)

Next, the sampling equipment 100 for the duodenal juice will be described with reference to FIG. 6.

The sampling equipment 100 shown in FIG. 6 is inserted into the body of the subject. The sampling equipment 100 collects the duodenal juice and stores the collected duodenal juice. For example, the sampling equipment described in the above-mentioned US Patent Application Publication No. 2012/0258478 can be used. A configuration of the sampling equipment 100 includes a catheter 102 for collection and a syringe 103. The syringe 103 is a collection container capable of storing therein the duodenal juice. The syringe 103 is connected to the catheter 102.

For example, an endoscopic retrograde cholangiopancreatography (ERCP) cannula (manufactured by Olympus Medical Systems Corporation; product name: PR-130Q) provided with two holes 104 in a radial direction on A tip side 102A can be used as the catheter 102.

The syringe 103 houses a gasket 106 and a plunger 107 within a cylindrical outer tube 105. The gasket 106 is slidable in the longer direction of the outer tube 105. The plunger 107 is connected to the gasket 106.

A flange 108 is connected to the plunger 107 in an end portion on the side opposite to the side on which the gasket 106 is connected. The flange 108 is capable of operating the gasket 106 so as to move. Furthermore, a nozzle 109 is attached to the outer tube 105. The nozzle 109 is inserted into the end portion of a base side 102B of the catheter 102. A protease inhibitor 111 is loaded in advance into a sealed space formed by the outer tube 105 and the gasket 106. Collected duodenal juice 112 is led through the catheter 102 into the syringe 103.

The duodenal juice 112 contains a large quantity of pancreatic enzyme activated by enterokinase in the duodenum. The pancreatic enzyme rapidly dissolves proteins and cells. Therefore, the protease inhibitor 111 (such as ethylenediaminetetraacetic acid [EDTA], aprotinin, phenylmethylsulfonyl fluoride [PMSF], or 4-(2-aminoethyl) benzenesulfonyl fluoride hydrochloride [AEBSF]) or a pH-control chemical reagent that suppresses pancreatic enzyme activity is preferably enclosed in advance, as the stabilizer for duodenal juice, in the syringe 103 connected to the base side 102B of the catheter 102.

As the collection container storing the duodenal juice 112, a collection trap set in front of the syringe may be used. In this instance, the protease inhibitor 111 may be enclosed in advance in the collection trap.

The duodenal juice 112 may be mixed with the protease inhibitor 111 at an early stage after being collected. In this case, the duodenal juice 112 may be transferred from the syringe to another storage container. In this instance, the protease inhibitor 111 may be enclosed in advance in the storage container to which the duodenal juice 112 is transferred from the syringe.

When collection of the duodenal juice 112 through the catheter 102 is started, the duodenal juice 112 is led into the syringe 103. Here, the protease inhibitor 111 is loaded into the syringe 103 in advance. Therefore, the duodenal juice 112 mixes with the protease inhibitor 111. Pancreatic enzyme activity can be instantly suppressed.

The protease inhibitor 111 may be in powder, solid or liquid form. The protease inhibitor 111 preferably mixes quickly with the collected duodenal juice 112 and dissolves. Depending on the type of protein, addition of the protease inhibitor 111 may not be required. However, for most proteins, detection accuracy is improved by the protease inhibitor 111 being added. In addition, the protease inhibitor 111 may be dispersed within the body. In this instance, a protease inhibitor that can be internally administered, such as Futhan or aprotinin that can injected intravenously, can be used.

### (Method for Collecting Duodenal Juice)

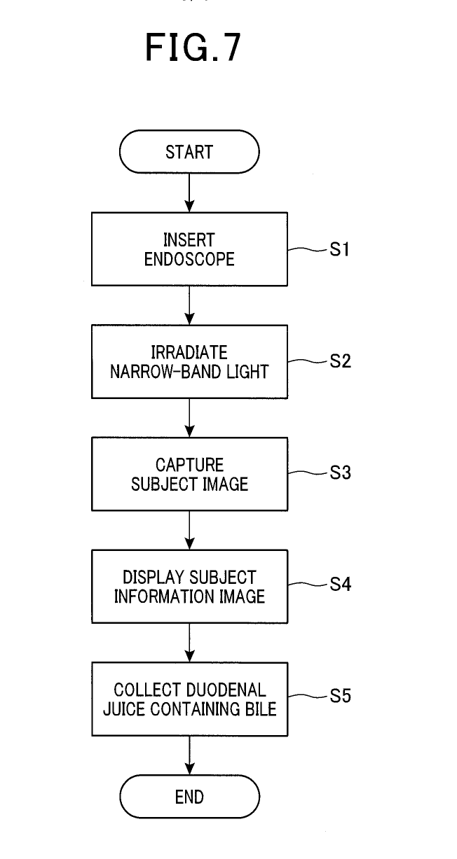

Next, procedures in the method for collecting duodenal juice from the subject using the endoscopy system 1 will be described for each step with reference to FIG. 7.

(1) First, the inserting section 301 of the electronic endoscope 3 is inserted into the duodenum of the subject from which the duodenal juice is to be collected (step S1).

According to the present embodiment, to detect pancreaticobiliary diseases in the subject, the inserting section 301 of the electronic endoscope 3 is inserted into an area from which duodenal juice containing large amounts of pancreatic juice and bile can be collected. This operation is performed in the normal light observation mode.

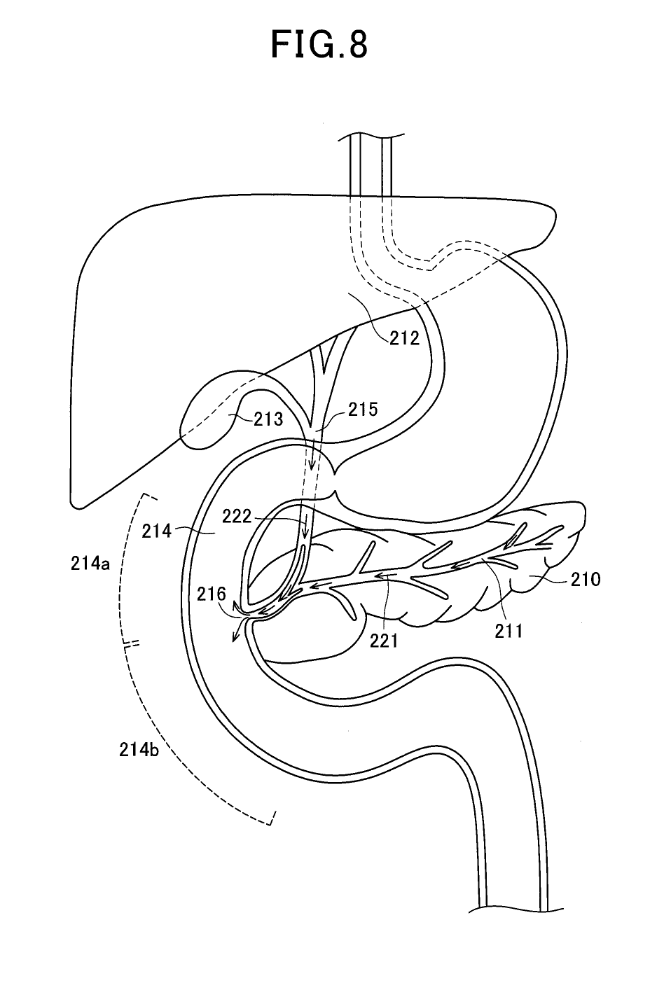

FIG. 8 describes the area from which the duodenal juice can be collected within the body.

As shown in FIG. 8, pancreatic juice 221 is produced and secreted by the pancreas 210. The pancreatic juice 221 accumulates in the pancreatic duct 211 running through the center of the pancreas 210. Bile 222 is secreted from the liver 212. The bile 222 is stored by the gall bladder 213 positioned on the lower surface of the liver 212 and accumulates in the common bile duct 215. The common bile duct 215 connects the gall bladder 213 and the duodenum 214.

Here, the pancreatic duct 211 in which the pancreatic juice 221 accumulates and the common bile duct 215 in which the bile 222 accumulates merge before the duodenum 214. Therefore, the pancreatic juice 221 in the pancreatic duct 211 and the bile 222 in the common bile duct 215 are secreted into the duodenum 214 through the papilla of Vater 216.

Therefore, the collection area of the duodenal juice is preferably near the papilla of Vater 216 from which the pancreatic juice 221 and the bile 222 are discharged. However, the position of the papilla of Vater 16 differs with the individual. The papilla of Vater 16 may be hidden behind a fold of mucosa or the like, and may not be visible in pictures. Therefore, at least either of a position 214a or a position 214b in the duodenum 214 shown in FIG. 8 is preferably set as the area from which the duodenal juice can be collected.

(2) Next, the tip portion of the inserting section 301 inserted into the duodenum is stopped inside the duodenum. In this state, the observation mode is switched to the narrow-band light observation mode. The green narrow-band light (wavelength band: 530 nm to 550 nm) and the blue narrow-band light (wavelength band: 390 nm to 445 nm) from the light-source device 4 are supplied to the inserting section 301 through the light guide 15. The green narrow-band light and the blue narrow-band light are then irradiated from the illumination lens 42 at the tip portion (step S2).

(3) Next, an image of the interior of the duodenum (subject image) that is illuminated by the green narrow-band light and the blue narrow-band light is captured by the CCD 2. The CCD 2 outputs the image signal of the captured subject image to the video processor 7 (step S3).

(4) Next, the video processor 7 generates the color image data capturing the interior of the duodenum (subject image) illuminated by the green narrow-band light and the blue narrow-band light based on the output signal from the CCD 2. The video processor 7 then displays the subject information image expressing the subject information based on the color image data on the observation monitor 5 (step S4).

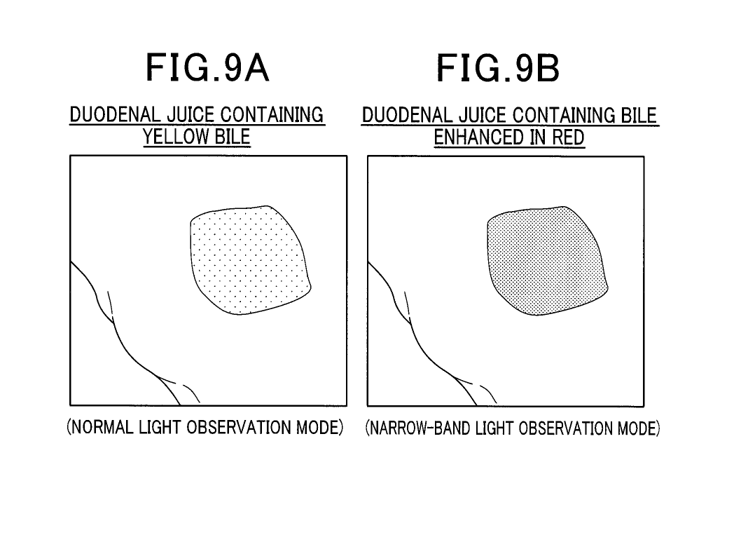

FIG. 9A shows the subject information image in the normal light observation mode, and FIG. 9B shows the subject information image in the narrow-band light observation mode for comparison.

As described above, bile contains bilirubin. Duodenal juice containing bile exhibits a light yellow color. Therefore, in the subject information image in the normal light observation mode shown in FIG. 9A, the bile contained in the duodenal juice is displayed as a light yellow fluid similar to that under macroscopic observation. Under an endoscope, the light yellow fluid is difficult to identify. Targeting and collecting the duodenal juice containing bile that exhibits the yellow color, while viewing the subject information image, is difficult.

On the other hand, as described above, the subject information image in the narrow-band light observation mode shown in FIG. 9B is composed of the color image data (R₁, G₁, and B₁) (refer to the above-described expression (1)). The color image data (R₁, G₁, and B₁) is generated by the image processing circuit 30 of the video processor 7 from the image data (G) obtained during irradiation of the green narrow-band light and the image data (B) obtained during irradiation of the blue narrow-band light.

According to the present embodiment, as shown in the above-described expression (2), the color image data (R₁) corresponding to red is calculated by the product of the image data (G) obtained during irradiation of the green narrow-band light and the coefficient h11(=1). The color image data (G₁) corresponding to green is calculated by the product of the image data (B) obtained during irradiation of the blue narrow-band light and the coefficient h22(=1.2). The color image data (B₁) corresponding to blue is calculated by the product of the image data (B) obtained during irradiation of the blue narrow-band light and the coefficient h32(=0.8).

Here, a fluid exhibiting a yellow color such as bile absorbs blue light, and reflects green and red lights. Therefore, bile reflects the green narrow-band light during irradiation of the green narrow-band light. The bile absorbs (barely reflects) the blue narrow-band light during irradiation of the blue narrow-band light.

Therefore, the intensity of returned light resulting from the green narrow-band light being reflected by bile is higher than the intensity of returned light resulting from the blue narrow-band light being reflected. Thus, in the above-described expression (2), the value of the color image data (R₁) corresponding to red that is calculated by the product of the image data (G) obtained during irradiation of the green narrow-band light and the coefficient h11 is greater than the values of the color image data (G₁) corresponding to green and the color image data (B₁) corresponding to blue that are respectively calculated by the products of the image data (B) obtained during irradiation of the blue narrow-band light and the coefficients h22 and h32.

As a result, in the subject information image in the narrow-band light observation mode shown in FIG. 9B, the bile contained in the duodenal juice is displayed such as to be enhanced in red as a whole (including reddish colors such as red to brownish-red), as a result of the color image data (R₁) corresponding to red. Under an endoscope, the red fluid can be easily identified. Targeting and selectively collecting the duodenal juice containing the bile that is displayed such as to be enhanced in red, while viewing the subject information image, is easy.

(5) Finally, the duodenal juice containing the bile that is displayed such as to be enhanced in red in the subject information image is collected, while observing the subject information image displayed on the observation monitor 5 (step S5).

According to the present embodiment, the above-described sampling equipment 100 shown in FIG. 6 is used to collect the duodenal juice. First, the catheter 102 of the sampling equipment 100 is inserted into the treatment tool insertion opening 312 of the manipulating section 302. The catheter 102 is then projected from the tip portion of the inserting section 301.

The duodenal juice containing the bile that is displayed such as to be enhanced in red in the subject information image on the observation monitor 5 is targeted. The tip side 102A of the catheter 102 is positioned in this targeted area. The duodenal juice is then selectively collected in the syringe 103 through the catheter 102.

Therefore, according to the present embodiment, the green narrow-band light and the blue narrow-band light that are illumination lights having specific wavelength bands are irradiated. As a result, the duodenal juice containing bile is displayed such as to be enhanced in red in the subject information image on the observation monitor 5. Therefore, the duodenal juice containing the bile that is displayed such as to be enhanced in red can be targeted and efficiently collected under an endoscope. The collection period of the duodenal juice can be shortened. As a result, the burden placed on the patient can be reduced. In addition, the duodenal juice containing bile can be efficiently collected. As a result, accuracy of biomolecule testing can be improved.

In the above-described endoscopy system 1, an endoscope device using a frame sequential method is described as an example. In the endoscope device using the frame sequential method, the light-source device 4 supplies the frame sequential light. The video processor 7 synchronizes the frame sequential image information and forms images. However, the endoscopy system 1 is not limited thereto. The present invention can also be applied to a synchronous endoscope device (for example, refer to JP-B2-4184626).

In addition, the spectral transmission characteristics of the second filter set of the rotary filter are not limited to those shown in FIG. 5. For example, the second filter set may have spectral transmission characteristics in which the spectral product of the green narrow-band light is less than the spectral product of the blue narrow-band light.

In addition, the second filter set of the rotary filter is composed of the G2 filter portion, the B2 filter portion, and the light-blocking filter portion in the example in FIG. 3. However, the second filter set is not limited thereto. For example, a B2 filter portion or a G2 filter portion may be disposed instead of the light-blocking filter portion.

In addition, h11=1, h12=0, h21=1, h22=1.2, h31=0, and h32=0.8 are given as examples of the values of the coefficients in above-described expression (1). However, the values are not limited thereto. For example, any value may be used as long as the duodenal juice containing bile can be displayed such as to be enhanced in red as a result of being irradiated with the above-described green narrow-band light and blue narrow-band light.

In addition, the above-described method for collecting duodenal juice may be applied to a method for collecting a yellow-colored fluid other than the duodenal juice containing bile. In this instance, the endoscope is inserted into a target site of the subject at which the yellow fluid is present. Illumination light of a specific wavelength band (such as the green narrow-band light and the blue narrow-band light) within the visible wavelength region is irradiated. As a result, the yellow fluid can be displayed such as to be enhanced in red in the subject information image on the observation monitor 5. As a result, the fluid can be targeted and collected upon recognition of the yellow fluid in the image.

In addition, the sampling equipment is not limited to that shown in FIG. 6. The sampling equipment is merely required to be that in which a catheter can be inserted into the forceps opening of an endoscope and the duodenal juice can be collected.

Furthermore, the collection container storing the duodenal juice is not limited to the syringe. For example, a collection trap set in front of the syringe, a collection bottle connected to a vacuum pump, or a absorber may be used. As a result of negative pressure being generated within the catheter by the collection container, the duodenal juice can be stored within the collection container

In addition, according to the present embodiment, the mode in which the green narrow-band light and the blue narrow-band light that are illumination lights of specific wavelength bands are irradiated is referred to as the “narrow-band light observation mode”. However, a device may be used in which the mode is referred to as a “duodenal juice collection mode”.

The present invention is not limited by the above-described embodiments and variation examples. Various embodiments in which the present invention is combined accordingly with known technology are also possible without departing from the spirit of the invention recited in the scope of claims.

What is claimed is:

1. A method for collecting duodenal juice, comprising:
a first step of inserting an endoscope into a duodenum of a subject from which collection of duodenal juice is performed;
a second step of generating an illumination light of a specific wavelength band, within a visible wavelength region, that is capable of displaying a component including bile in an enhanced manner, the illumination light of the specific wavelength band being generated in a state in which an illuminating section of the endoscope is stopped at the duodenum;
a third step of capturing a subject image that is illuminated by the illumination light of the specific wavelength band, the subject image being captured by an imaging section of the endoscope;
a fourth step of displaying a subject information image in a display section based on an output signal from the imaging section, the subject information image expressing subject information; and
a fifth step of collecting duodenal juice containing the bile that is displayed in an enhanced manner in the subject information image displayed in the display section.

2. The method according to claim 1, wherein the specific wavelength band includes a first wavelength band of 390 nm to 445 nm and a second wavelength band of 530 nm to 550 nm.

3. The method according to claim 2, wherein, in the fifth step, the duodenal juice containing the bile that is enhanced in red in the displayed subject information image is collected.
